(19) United States
(12) Patent Application Publication
(10) Pub. No.: US 2025/0241914 A1
(43) Pub. Date: Jul. 31, 2025

(54) BIFUNCTIONAL PHARMACEUTICAL COMPOUNDS

(71) Applicant: DE MONTFORT UNIVERSITY, Leicester (GB)

(72) Inventor: Federico BRUCOLI, Leicester (GB)

(21) Appl. No.: 19/041,199

(22) Filed: Jan. 30, 2025

(30) Foreign Application Priority Data
Jan. 31, 2024 (GB) 2401279.1

(51) Int. Cl. A61K 31/513; A61P 31/06; C07D 405/14

(52) U.S. Cl. CPC A61K 31/513; A61P 31/06; C07D 405/14

(57) ABSTRACT

A bifunctional compound for use as a medicament. The compound comprises a rifamycin or a rifamycin derivative moiety; a linker; and a nucleobase analogue or a nucleoside analogue moiety.

### TECHNOLOGICAL FIELD

Embodiments of the present disclosure relate to bifunctional compounds for use as a medicament, and in particular to bifunctional compounds comprising a first rifamycin or rifamycin derivative moiety along with a second nucleobase analogue or a nucleoside analogue moiety.

### BACKGROUND

The overuse of antibiotics has led to a surge in drug-resistant pathogen strains, rendering many conventional treatments ineffective. For instance, prosthetic joint infections (PJIs) can occur after a joint replacement, and are expected to become more common due to antibiotic resistance and a rise in the number of joint replacement surgeries. In the UK alone between 2005 and 2023, there was a 4.6-fold rise in PJI cases. PJIs are commonly caused by Staphylococcus aureus and coagulase negative staphylococci (CoNS), and are usually treated with an intensive combination therapy including antibiotics and debridement. Such treatments are a burden on the healthcare system and are sometimes ineffective.

Tuberculosis is commonly treated with a combination of antibiotic drugs including rifampicin, which is an iminomethyl derivative of rifamycin SV, to minimise the risk of drug resistance. However, drug-resistant tuberculosis, and in particular rifampicin-resistant tuberculosis, is also becoming more prevalent.

There is, therefore, a need to provide new effective and safe treatments and/or prophylaxis for such diseases.

### BRIEF SUMMARY

According to various, but not necessarily all, embodiments there is provided a compound with the general structure of formula I, or a pharmaceutically acceptable salt, solvate, or prodrug thereof

R¹-L-R² (Formula I)
wherein
R¹ is a rifamycin or a rifamycin derivative;
L is a linker;
R² is a nucleobase analogue or a nucleoside analogue; and
the compound is for use as a medicament.

The nucleobase analogue or nucleoside analogue may comprise a purine analogue moiety or a pyrimidine analogue moiety.

The nucleobase analogue or nucleoside analogue may be thioguanine, 6-mercaptopurine, 6-thioguanosine, 5-fluorouracil, 5-fluorocytosine, emtricitabine, 2′-deoxycytidine, floxuridine, 5-fluorouridine, trifluridine, 2′, 3′-dideoxyinosine, gemcitabine, zidovudine or idoxuridine. The nucleoside analogue may be a ribonucleoside or deoxyribonucleoside modified with an alkoxy group at the 2′ or 3′ position of the ribose moiety. The alkoxy group may be a methoxy group.

The nucleobase analogue or nucleoside analogue may be 5-fluorouracil or emtricitabine.

The rifamycin or rifamycin derivative may have the general formula II or the general formula III.

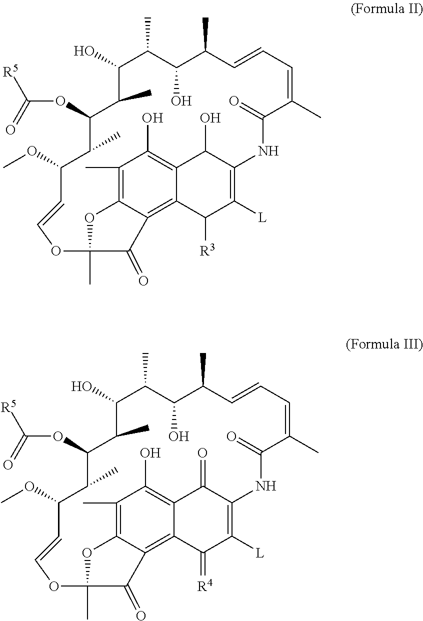

wherein
R³ is selected from an OH group, an NH₂ group, a carboxymethoxy group, or a heterocycle formed with the linker;
R⁴ is selected from an O, an NH group, or a heterocycle formed with the linker;
R⁵ is selected from a CH₃ group, a secondary amine or a tertiary amine; and
L indicates the attachment to the linker.

The rifamycin or rifamycin derivative may have the general formula IV

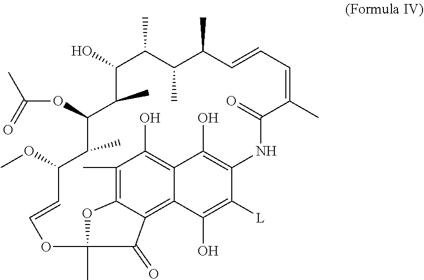

wherein:
L indicates the attachment to the linker.

The linker may be configured to substantially prevent steric hindrance between R¹ and R². The linker may be an organic chain with a length of 3 to 50 atoms. The linker may be an organic chain with a length of 5 to 30 atoms. The linker may be an organic chain with a length of at least 12 atoms.

The linker may comprise: a linear or branched alkyl chain; or an ethylene glycol chain.

The linker may have the general formula V

R¹-L¹-L²-L³-R² (Formula V)
wherein
R¹ indicates attachment to R¹;
R² indicates attachment to R²;
L¹ is selected from:

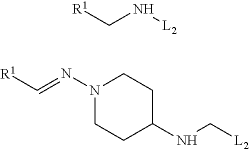

wherein R¹ indicates attachment to R¹ and L² indicates attachment to L²
L² is an organic chain with a length of 1 to 50 atoms; and
L³ is selected from:

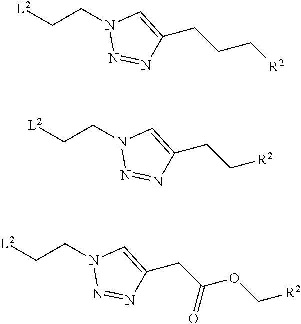

wherein L² indicates attachment to L² and R² indicates attachment to R².
L² may be a linear or branched alkyl chain; or an ethylene glycol chain.

The compound may be selected from:

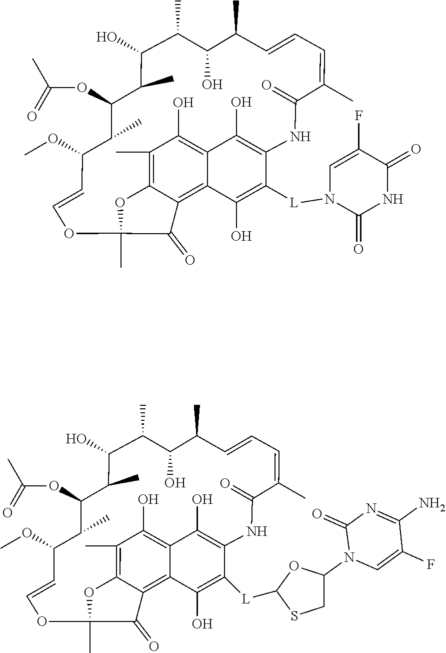

or a pharmaceutically acceptable salt, solvate, or prodrug thereof, wherein L is the linker.

The compound may be selected from:

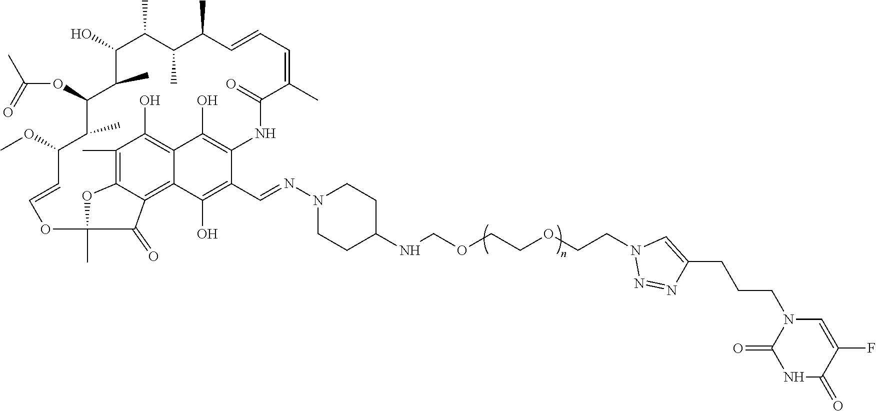

wherein n is from 1 to 10

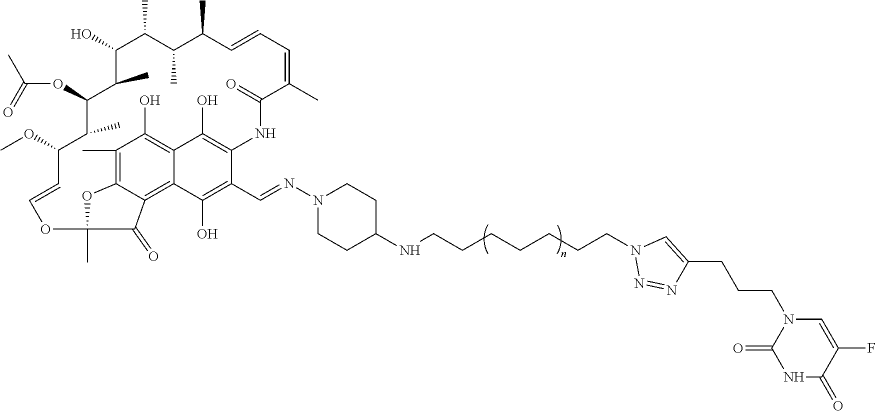

wherein n is from 0 to 10

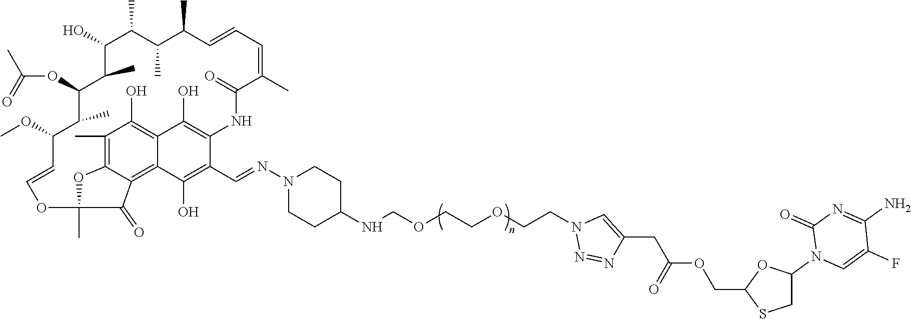

wherein n is from 1 to 10

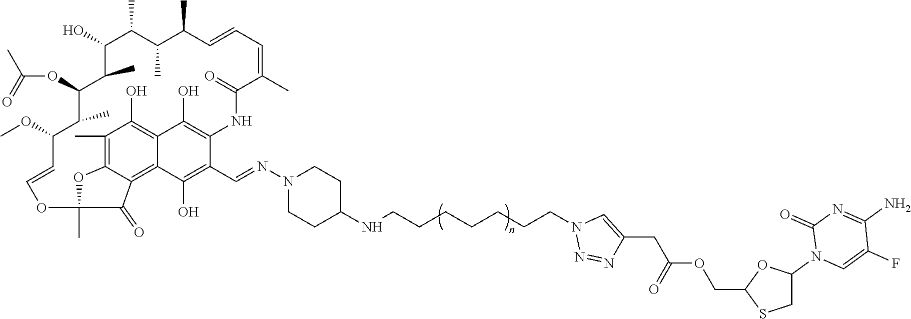

wherein n is from 0 to 10

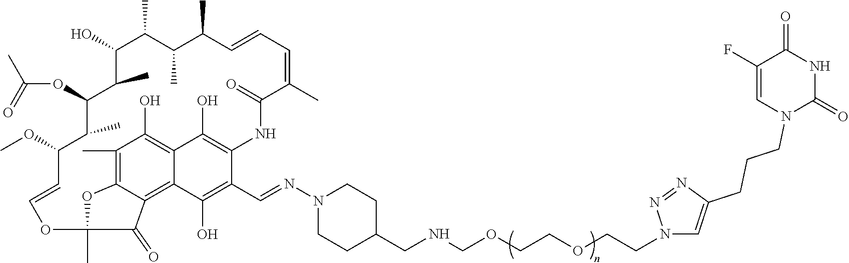

wherein n is from 1 to 10

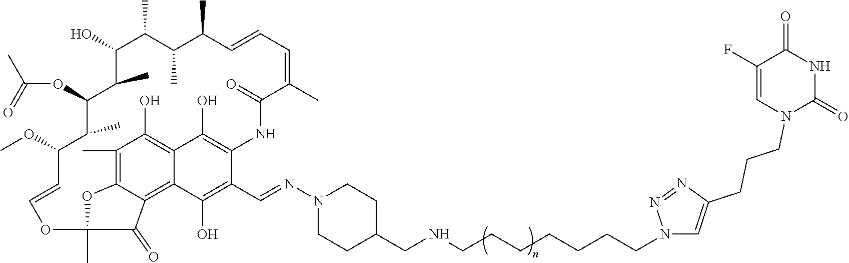

wherein n is from 0 to 10

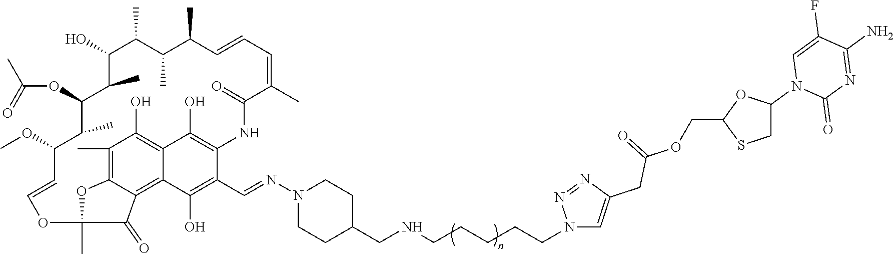

wherein n is from 0 to 10

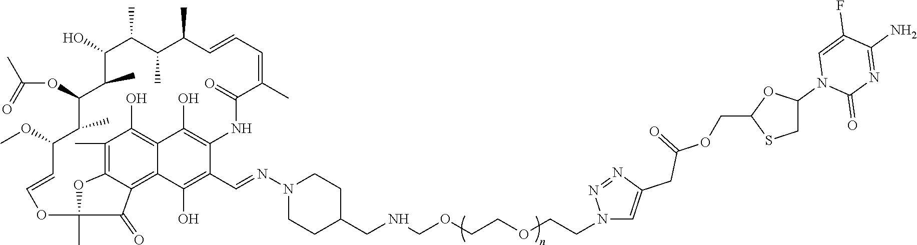

wherein n is from 1 to 10
or a pharmaceutically acceptable salt, solvate, or prodrug thereof.

The compound may be selected from:

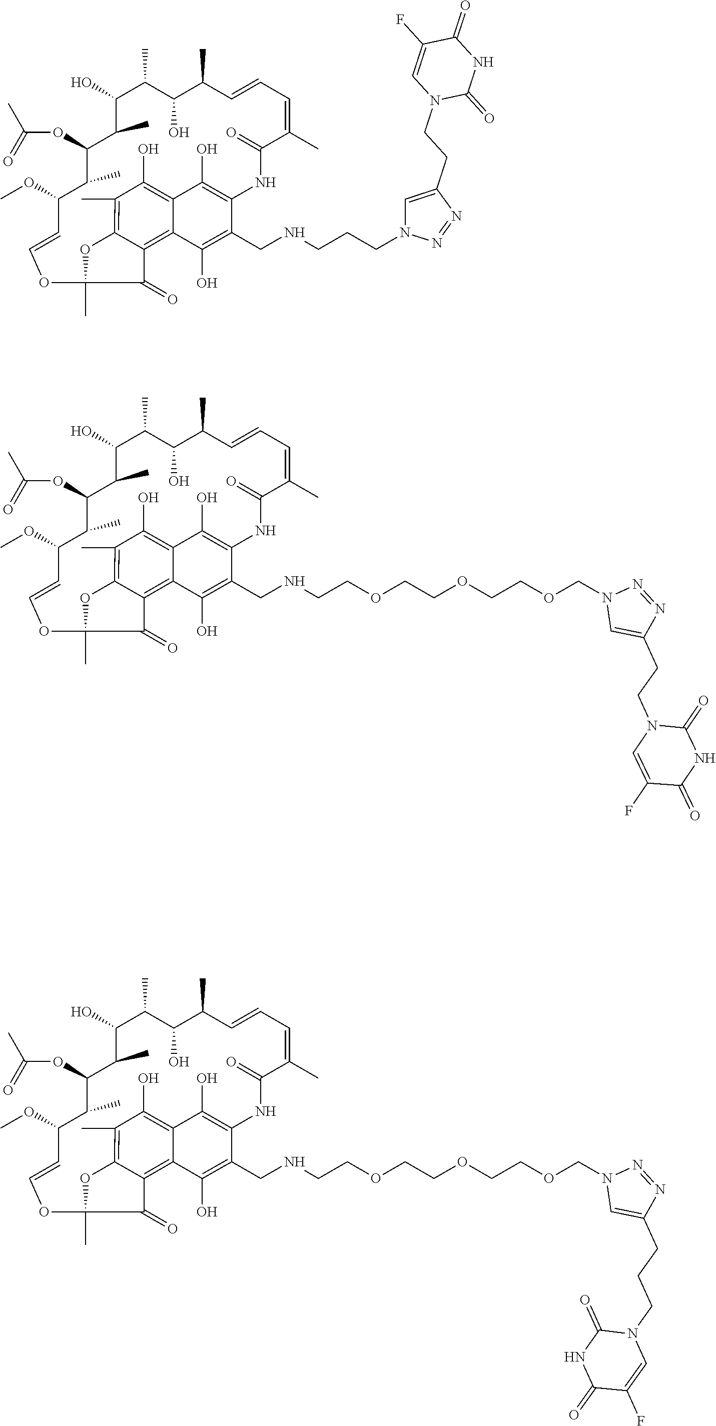

or a pharmaceutically acceptable salt, solvate, or prodrug thereof.

Possibly, the compound is for use in the treatment of an infection or prevention of an infection. The infection may be a bacterial infection. Possibly, the compound is for use as an antibiotic.

Possibly, the compound is for use in the treatment of tuberculosis. Possibly, the compound is for use in the treatment of prosthetic joint infections (PJIs).

According to various, but not necessarily all, embodiments there is provided a pharmaceutical composition which comprises a compound according to any of the preceding paragraphs, or a pharmaceutically acceptable salt, solvate or prodrug thereof, in association with a pharmaceutically acceptable diluent or carrier.

According to various, but not necessarily all, embodiments there is provided a method of treating or preventing an infection in an individual, the method comprising the step of administering to the individual a therapeutically effective amount of the compound of any of the preceding claims. Possibly, the method is a method of treating tuberculosis. Possibly, the method is a method of treating prosthetic joint infections (PJIs).

According to various, but not necessarily all, embodiments there is provided examples as claimed in the appended claims.

### BRIEF DESCRIPTION

For a better understanding of various examples that are useful for understanding the detailed description, reference will now be made by way of example only.

### DETAILED DESCRIPTION

The disclosure provides compounds with the general structure of formula I, and pharmaceutically acceptable salts, solvates, or prodrugs thereof.

R¹-L-R² (Formula I)
R¹ is a rifamycin or a rifamycin derivative;
L is a linker; and
R² is a nucleobase analogue or a nucleoside analogue.

The disclosure also provides a pharmaceutical composition which comprises the compound, or a pharmaceutically acceptable salt, solvate or prodrug thereof, in association with a pharmaceutically acceptable diluent or carrier.

The compounds are for use as a medicament. The compounds are for use in the treatment or prophylaxis/prevention of microbial infection, such as a bacterial infection.

The disclosure provides a method of treating or preventing an infection in an individual, such as a bacterial infection, the method comprising the step of administering to the individual a therapeutically effective amount of the compound described herein. In some examples the compound described herein may be administered in combination with one or more other compounds useful for the treatment or prevention of infection.

Target bacteria includes (but is not limited to) Mycobacterium tuberculosis (i.e., tuberculosis), Staphylococcus aureus and coagulase negative staphylococci (CoNS).

Without being bound by theory, it is believed that the therapeutic mode of action includes the rifamycin or rifamycin derivative moiety serving as an RNA polymerase targeted antibacterial carrier, which delivers the nucleoside analogue or nucleobase analogue moiety to a target that is in the vicinity of, but different to, the target of the rifamycin or rifamycin derivative. The analogue or nucleobase analogue moiety can act as an antimetabolite.

Compounds according to the disclosure may be orally bioavailable. Possible routes of administration include oral administration, intramuscular injection, intravenous injection, rectal administration, and other forms of administration.

### Rifamycin or Rifamycin Derivative

R¹ of Formula I is a rifamycin or a rifamycin derivative. A rifamycin derivative can also be referred to as a rifalogue. When part of the compounds described herein, the rifamycin or a rifamycin derivative can also be referred to as a rifamycin or a rifamycin derivative moiety. The rifamycin or rifamycin derivative is bonded to the linker.

Example rifamycins or rifamycin derivatives are shown in Table 1 below. The positions of the rifamycin system described herein are labelled on example 1C of Table 1. Positions 38 to 42 are specific to example 1C of Table 1, and do not apply to other examples described herein. The examples of Table 1 illustrate example rifamycin or rifamycin derivatives prior to attachment to a linker and formation of the compound of Formula I. It is to be understood that in the compounds described herein, the rifamycin or rifamycin derivative is connected to the linker, therefore substituents on for instance the 3 position in the examples of Table 1 may form part of the linker in the compounds described herein, when the linker is bonded to the rifamycin or rifamycin derivative at the 3 position. In other examples, the linker may be bonded to the rifamycin or rifamycin derivative at the 25 position.

TABLE 1

| ID | Structure |
|---|---|
| 1A | 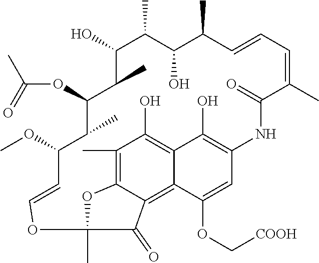 |
| | Rifamycin B |
| 1B | 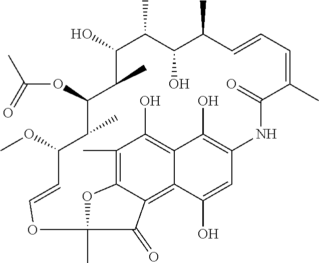 |
| | Rifamycin SV |
| 1C | 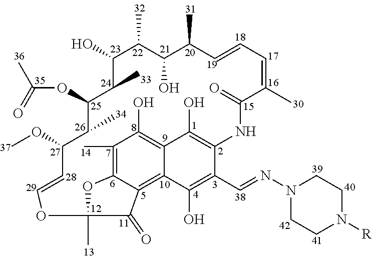 |
| | Rifampicin where R = CH₃ |
| | Rifapentine where R = cyclopentane |
| 1D | 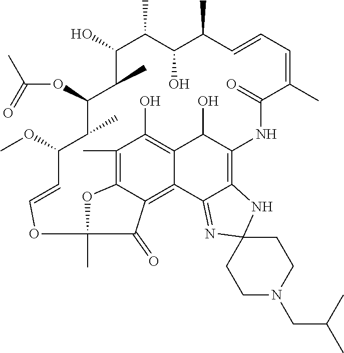 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(C)=O)[C@@H]1C)C4O] |
| | Rifabutin |
| 1E | 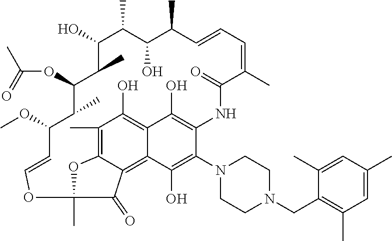 |
| | CGP-7040 |
| 1F | 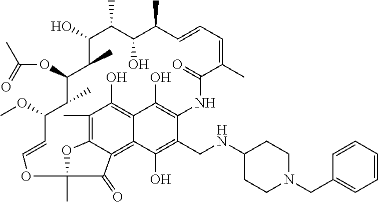 [SMILES: CO[C@H]1/C=C/O[C@@]2(C)Oc3c(C)c(O)c4c(O)c(c(CNC5CCN(Cc6ccccc6)CC5)c(O)c4c3C2=O)NC(=O)/C(C)=C\C=C\[C@H](C)[C@H](O)[C@@H](C)[C@@H](O)[C@@H](C)[C@H](OC(C)=O)[C@@H]1C] |
| | Rifamycin analogue 1F |
| 1G | 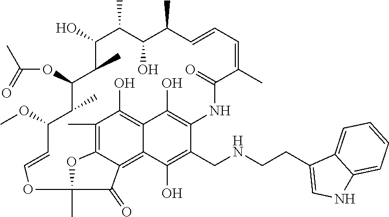 [SMILES: CO[C@H]1/C=C/O[C@@]2(C)Oc3c(C)c(O)c4c(O)c(c(CNCCc5c[nH]c6ccccc56)c(O)c4c3C2=O)NC(=O)/C(C)=C\C=C\[C@H](C)[C@H](O)[C@@H](C)[C@@H](O)[C@@H](C)[C@H](OC(C)=O)[C@@H]1C] |
| | Rifamycin analogue 1G |
| 1H | 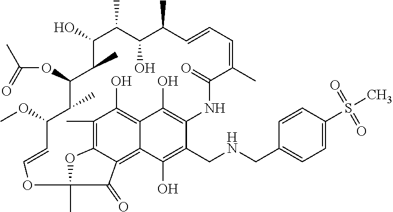 |
| | Rifamycin analogue 1H |
| 1I | 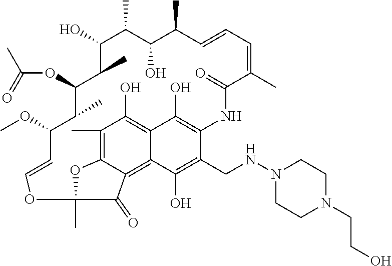 [SMILES: CO[C@H]1/C=C/O[C@@]2(C)Oc3c(C)c(O)c4c(O)c(c(CNN5CCN(CCO)CC5)c(O)c4c3C2=O)NC(=O)/C(C)=C\C=C\[C@H](C)[C@H](O)[C@@H](C)[C@@H](O)[C@@H](C)[C@H](OC(C)=O)[C@@H]1C] |
| | Rifamycin analogue 1I |
| 1J | 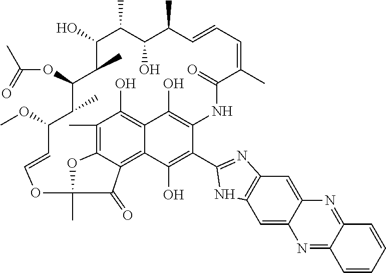 [SMILES: CO[C@H]1/C=C/O[C@@]2(C)Oc3c(C)c(O)c4c(O)c(c(-c5nc6cc7nc8ccccc8nc7cc6[nH]5)c(O)c4c3C2=O)NC(=O)/C(C)=C\C=C\[C@H](C)[C@H](O)[C@@H](C)[C@@H](O)[C@@H](C)[C@H](OC(C)=O)[C@@H]1C] |
| | Rifaphenazine |

In some examples the rifamycin or rifamycin derivative has the general formula II or the general formula III.

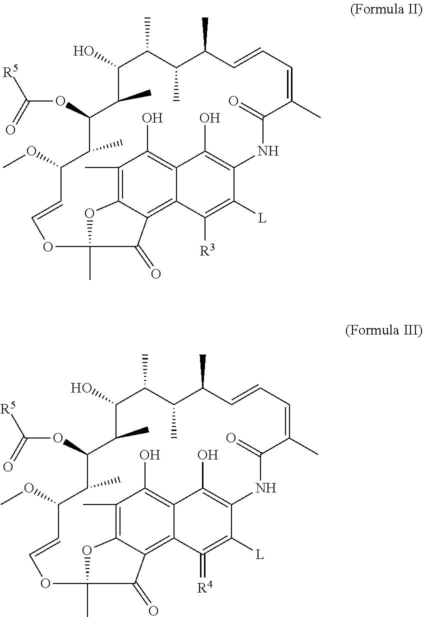

wherein
R³ is selected from an OH group, an NH₂ group, a carboxymethoxy group, or a heterocycle formed with the linker;
R⁴ is selected from an O, an NH group, or a heterocycle formed with the linker;
R⁵ is selected from a CH₃ group, a secondary amine or a tertiary amine; and
L indicates the attachment to the linker.

A heterocycle refers to a ring system in which one or more ring atoms is a heteroatom, e.g., nitrogen, oxygen, or sulfur (i.e., not carbon). R⁴ could for instance form an imidazoline ring or an imidazole ring in combination with the linker.

A carboxymethoxy group refers to an —O—CH₂—COOH group, as shown for instance at position 4 of example 1A in Table 1. The positions of the rifamycin system described herein are labelled on structure 1C of Table 1. Positions 38 to 42 are specific to structure 1C of Table 1, and do not apply to other examples described herein.

In some examples, the rifamycin or rifamycin derivative has the general formula IV

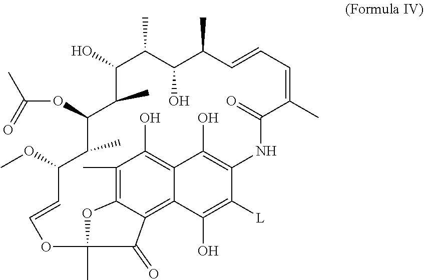

wherein:
L indicates the attachment to the linker.

### Nucleobase Analogue or Nucleoside Analogue

R² of Formula I is a nucleobase analogue or a nucleoside analogue. The nucleobase analogue or nucleoside analogue may act as an antimetabolite.

A nucleobase, which can also be referred to as a nitrogenous base, is a nitrogen-containing heterocyclic moiety capable of forming Watson-Crick-type hydrogen bonds to form a base pair with a complementary nucleobase or nucleobase analogue. In many examples, nucleobases are purines or pyrimidines. A nucleobase analogue is naturally not present in the human body but is sufficiently structurally similar to a nucleobase occurring naturally in the human body, such that it can substitute for the nucleobase occurring naturally in the human body during DNA or RNA synthesis. A nucleobase occurring naturally in the human body can be considered a normal nucleobase, which includes cytosine, guanine, adenine, thymine, and uracil. A nucleobase analogue may comprise an antimetabolite, which inhibits the use of a metabolite.

A nucleoside comprises a sugar residue (the sugar residue is often pentose) which is linked to a nucleobase via a glycosidic bond. A nucleoside analogue is naturally not present in the human body but is sufficiently structurally similar to a nucleoside occurring naturally in the human body, such that it can substitute for the nucleoside occurring naturally in the human body or in prokaryotic cells during DNA or RNA synthesis. A nucleoside analogue can substitute for a normal nucleoside in RNA or DNA synthesis. A nucleoside occurring naturally in the human body can be considered a normal nucleoside, which includes adenosine, guanosine, 5-methyluridine, uridine, cytidine, deoxyadenosine, deoxyguanosine, thymidine, deoxyuridine and deoxycytidine. A nucleobase analogue may comprise an antimetabolite, which inhibits the use of a metabolite. The nucleoside analogue may, once phosphorylated, be incorporated into a DNA strand, and can terminate the DNA chain.

The nucleobase analogue or nucleoside analogue may comprise a purine analogue or a pyrimidine analogue. Purine analogues mimic the structure of metabolic purines, and pyrimidine analogues mimic the structure of metabolic pyrimidines. In some examples, the nucleobase analogue or nucleoside analogue is a fluorinated pyrimidine or a thiopurine.

In some examples, the nucleobase analogue or nucleoside analogue is thioguanine, 6-mercaptopurine, 6-thioguanosine, 5-fluorouracil, 5-fluorocytosine, emtricitabine, 2′-deoxycytidine, floxuridine, 5-fluorouridine, trifluridine, 2′, 3′-dideoxyinosine, gemcitabine, zidovudine or idoxuridine. The nucleoside analogue may be a ribonucleoside or deoxyribonucleoside modified with an alkoxy group at the 2′ or 3′ position of the ribose moiety. The alkoxy group may be a methoxy group.

Where the compound includes a nucleobase analogue comprising a purine analogue, the nucleobase analogue may be connected to the linker at position 2 or 9 of the purine analogue ring. Where the compound includes an adenine analogue, the adenine analogue may be connected to the linker at the amine group attached to position 6 of the adenine analogue ring. Where the compound includes a guanine analogue, the guanine analogue may be connected to the linker at the amine group attached to position 2 of the guanine analogue ring.

Where the compound includes a nucleobase analogue comprising a pyrimidine analogue, the nucleobase analogue may be connected to the linker at position 1 of the pyrimidine analogue ring. Where the compound includes a nucleoside analogue with a pentose ring or pentose analogue ring, the nucleobase analogue may be connected to the linker at position 5′ of the pentose ring or pentose analogue ring. The ring positions of nucleobase analogues and nucleoside analogues described herein are as determined by the International Union of Pure and Applied Chemistry (IUPAC) rules and guidelines.

### Linker

R¹ and R² of Formula I are linked by a linker. The linker links the rifamycin or rifamycin derivative to the nucleobase analogue or the nucleoside analogue. A linker can be any suitable moiety that spaces R¹ from R².

In some examples, the linker is an organic chain with a length of 3 to 50 atoms. The linker may be an organic chain with a length of 5 to 30 atoms. The linker may be an organic chain with a length of at least 7 atoms or at least 12 atoms.

The linker may be of sufficient length to substantially prevent interaction between R¹ and R². The linker may be of sufficient length to substantially prevent steric hindrance between R¹ and R². In some examples, the linker is at least 10 Å in length. The linker may at least 15 Å in length, or preferably at least 20 Å in length. The linker may be between 20 Å and 80 Å in length.

The organic chain could be unsaturated or saturated. The organic chain may include for instance one or more cyclic groups, such as a heterocycle, a cycloalkane, or aryl groups. The organic chain may include side chains, such as alkyl, alkenyl, or aryl side chains. The organic chain may include one or more functional groups such as ether groups, carboxy groups, hydroxy groups, halide groups, etc. The organic chain may include one or more heteroatoms along the chain, for instance the chain may include one or more polyethylene glycol units.

In some examples, the linker comprises a linear or branched alkyl chain or an ethylene glycol chain.

Preferably, the linker comprises an ethylene glycol chain (i.e., a (CH₂CH₂O)ₙ moiety), otherwise known as polyethylene glycol (PEG). The ethylene glycol chain may have an average (mean) molecular mass of from 150 g/mol to 2500 g/mol. For instance, the linker may be formed from functionalised PEG-4, functionalised PEG-400 or functionalised PEG-2000.

In many examples, prior to forming a compound with R¹ and R², precursor to the linker (hereafter referred to as the linker precursor) contains at least two functional groups, one of which reacts with R¹ and the other reacts with R². Once the linker precursor has reacted with R¹ and R² to form the compound, the term linker refers to the part of the resulting structure between R¹ and R². The linker precursor can comprise a portion that does not participate in a bond with the entities being linked, and whose main purpose may be to spatially separate the entities from each other, which is referred to as a spacer herein.

In some examples, the spacer is a linear or branched alkyl chain or an ethylene glycol chain. The spacer may comprise one or more cyclic moieties, such as a heterocycle or a cycloalkane.

Preferably, the spacer is an ethylene glycol chain (i.e., a (CH₂CH₂O)ₙ moiety), otherwise known as polyethylene glycol (PEG). The ethylene glycol chain may have an average (mean) molecular mass of from 150 g/mol to 2500 g/mol. For instance, the spacer may be formed from functionalised PEG-4, functionalised PEG-400 or functionalised PEG-2000.

In some examples, the linker has the general formula V

R¹-L¹-L²-L³-R² (Formula V)
wherein
R¹ indicates attachment to R¹;
R² indicates attachment to R²;
L¹ is selected from:

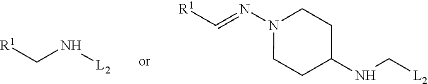

wherein R¹ indicates attachment to R¹ and L² indicates attachment to L²
L² is an organic chain with a length of 1 to 50 atoms; and
L³ is selected from:

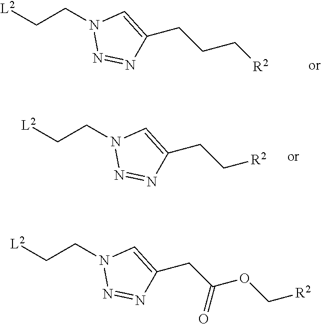

wherein L² indicates attachment to L² and R² indicates attachment to R².

L² may be an organic chain with a length of 1 to 40 atoms. L² may be an organic chain with a length of 1 to 15 atoms. L² may be an organic chain with a length of at least 3 atoms or at least 5 atoms.

The organic chain could be unsaturated or saturated. The organic chain may include for instance one or more cyclic groups, such as a heterocycle, a cycloalkane, or aryl groups. The organic chain may include side chains, such as alkyl, alkenyl, or aryl side chains. The organic chain may include one or more functional groups such as ether groups, carboxy groups, hydroxy groups, halide groups, etc. The organic chain may include one or more heteroatoms along the chain, for instance the chain may include one or more polyethylene glycol units.

In some examples, L² comprises a linear or branched alkyl chain or an ethylene glycol chain.

Preferably, the L² is an ethylene glycol chain (i.e., a (CH₂CH₂O)ₙ moiety), otherwise known as polyethylene glycol (PEG). The ethylene glycol chain may have an average (mean) molecular mass of from 150 g/mol to 2500 g/mol. For instance, L² may be formed from PEG-4, PEG-400 or PEG-2000.

Examples of compounds according to the disclosure with linkers are provided in Table 2 below. The linkers of Table 2 vary in length depending on the chosen value of n.

TABLE 2

| ID | Structure |
|---|---|
| 2A | 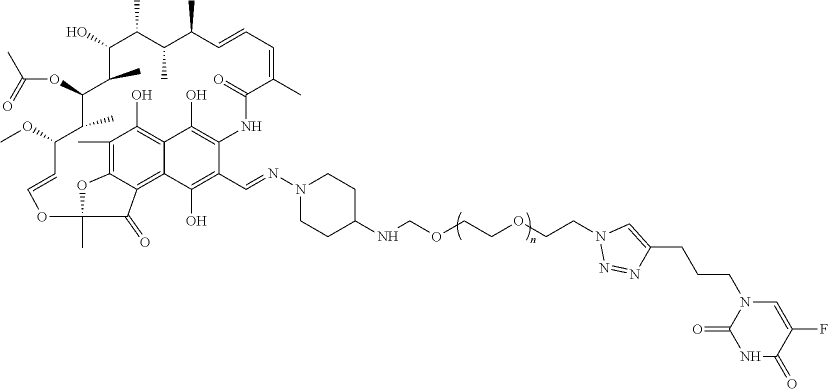 |
| 2B | 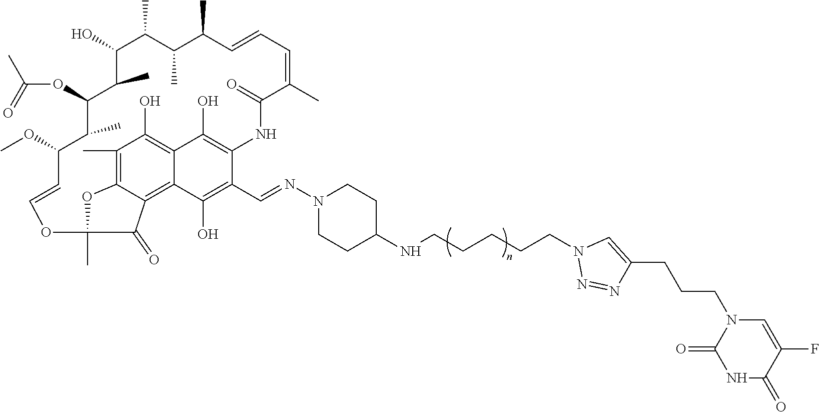 |
| 2C | 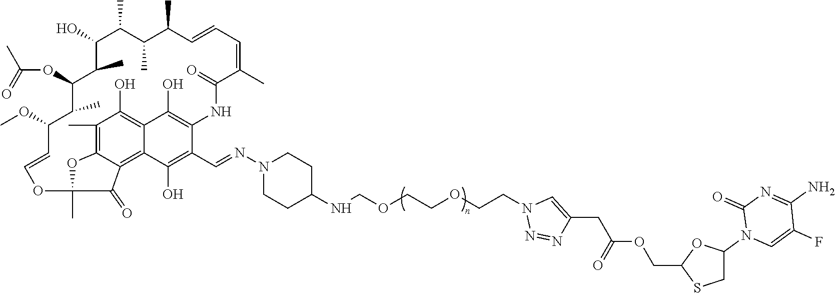 |
| 2D | 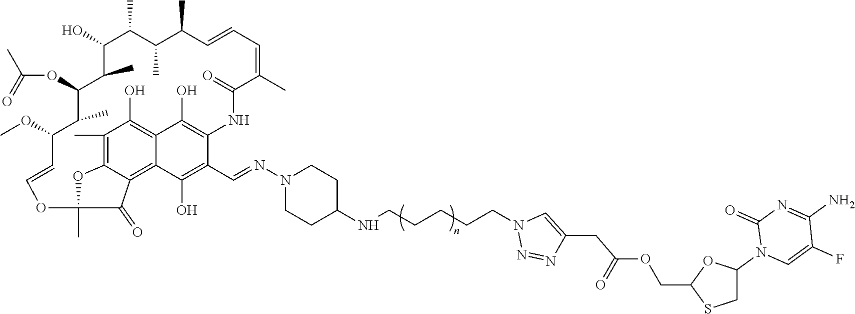 |
| 2E | 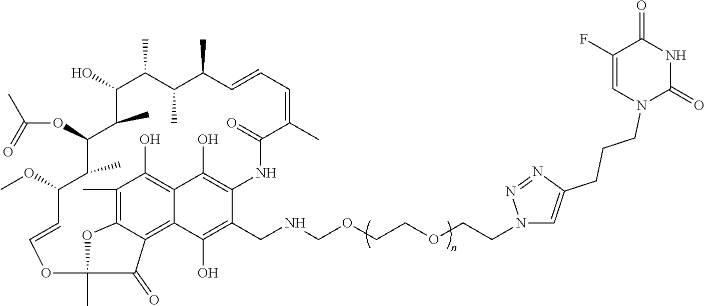 |
| 2F | 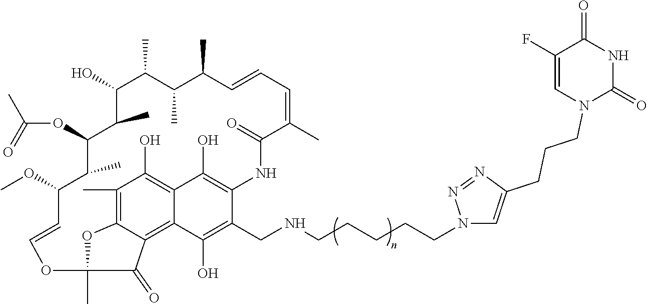 |
| 2G | 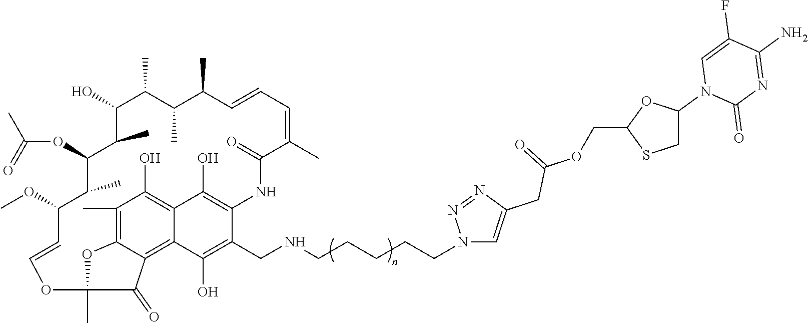 |
| 2H | 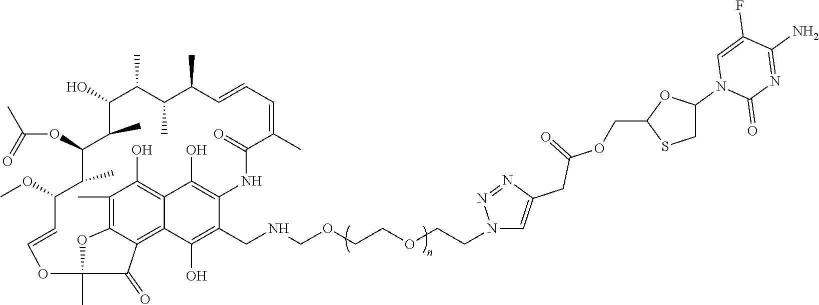 |

In examples 2B, 2D, 2F and 2G, n may be between 0 and 10. Preferably, n is between 0 and 5.

In examples 2A, 2C, 2E and 2H, n may be between 1 and 10. Preferably, n is between 1 and 5.

Further examples of compounds according to the disclosure are provided in Table 3 below.

TABLE 3

| ID | Structure |
|---|---|
| 3A | 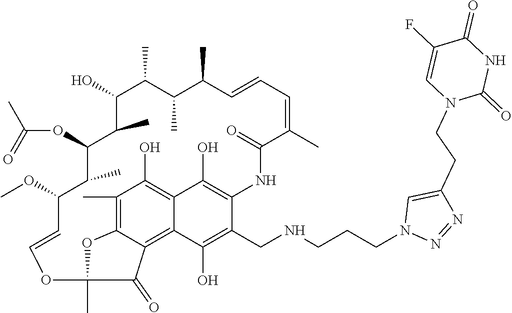 [SMILES: CO[C@H]1/C=C/O[C@@]2(C)Oc3c(C)c(O)c4c(O)c(c(CNCCCn5cc(CCn6cc(F)c(=O)[nH]c6=O)nn5)c(O)c4c3C2=O)NC(=O)/C(C)=C\C=C\[C@H](C)[C@H](C)[C@@H](C)[C@@H](O)[C@@H](C)[C@H](OC(C)=O)[C@@H]1C] |
| 3B | 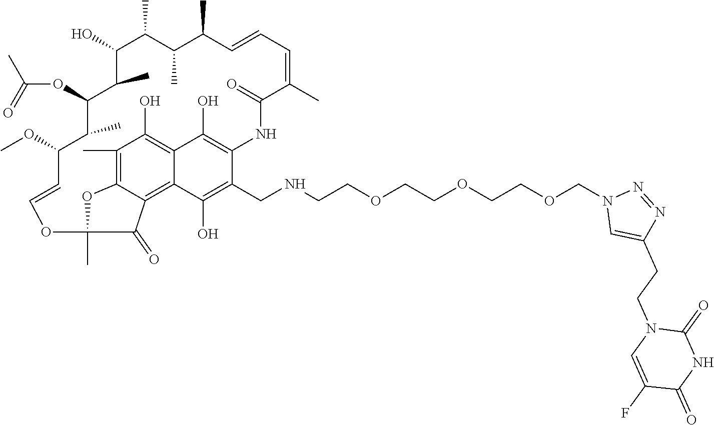 [SMILES: CO[C@H]1/C=C/O[C@@]2(C)Oc3c(C)c(O)c4c(O)c(c(CNCCOCCOCCOCn5cc(CCn6cc(F)c(=O)[nH]c6=O)nn5)c(O)c4c3C2=O)NC(=O)/C(C)=C\C=C\[C@H](C)[C@H](C)[C@@H](C)[C@@H](O)[C@@H](C)[C@H](OC(C)=O)[C@@H]1C] |
| 3C | 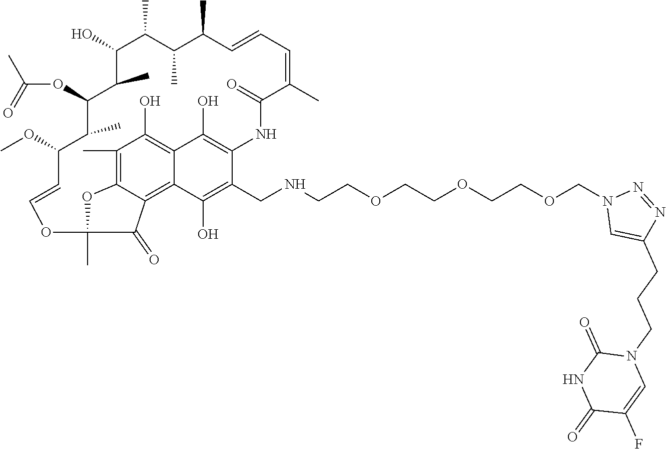 [SMILES: CO[C@H]1/C=C/O[C@@]2(C)Oc3c(C)c(O)c4c(O)c(c(CNCCOCCOCCOCn5cc(CCCn6cc(F)c(=O)[nH]c6=O)nn5)c(O)c4c3C2=O)NC(=O)/C(C)=C\C=C\[C@H](C)[C@H](C)[C@@H](C)[C@@H](O)[C@@H](C)[C@H](OC(C)=O)[C@@H]1C] |

### Preparation

Example preparations of compounds according to the disclosure are now described.

In some examples, the preparation of the compounds according to the disclosure is carried out in multiple steps. The compound may be made from a number of building blocks. One of the steps may include the reaction of a rifamycin block or rifamycin derivative block with a linker precursor, to form an intermediate. A further step is the reaction of a nucleobase analogue block or nucleoside analogue block with the intermediate. The “rifamycin block or rifamycin derivative block” is hereafter referred to as the RIF block, and the “nucleobase analogue block or nucleoside analogue block” is hereafter referred to as the NUC block.

Reaction of the RIF Block with the Linker Precursor to Form an Intermediate

An example of a reaction of a RIF block with a linker precursor to form an intermediate is shown in Scheme 1 below.

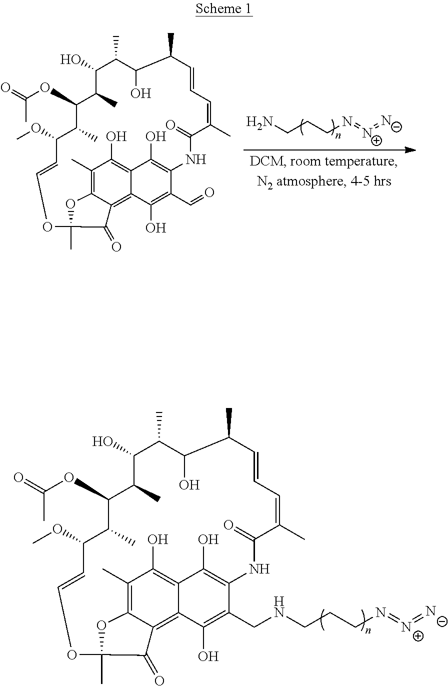

A linker precursor with a first amino functional group and a second azide functional group (1 equivalent) was added to a round bottom flask containing 10 mL of dichloromethane in which 3-formyl rifamycin SV (1 equivalent) was previously dissolved. The solution was then treated with sodium triacetoxyborohydride (1.4 equivalents), and stirred at room temperature under an N₂ atmosphere, until the consumption of the starting materials was observed by thin layer chromatography. At which point the reaction was neutralised through the addition of 10 mL 10% sodium bicarbonate. Ethyl acetate was then used to extract the product, and the organic layers were collected and washed with brine. Magnesium sulphate was used to dry the extracts, which was then filtered and concentrated under reduced pressure.

In the example preparation above, the linker precursor comprises a first functional group in the form of an amine functional group, and a second functional group in the form of an azido functional group. In other examples, the first functional group may be any other functional group suitable for reacting with (i.e., coupling to) the RIF block, and the second functional group may be any other functional group suitable for reacting with (i.e., coupling to) the NUC block.

In the example preparation above, the linker precursor comprises an alkyl chain between the first functional group and the second functional group. In other examples, the linker precursor may comprise a different organic chain. The organic chain could be unsaturated or saturated. The organic chain may include for instance one or more cyclic groups, such as a heterocycle, a cycloalkane, or aryl groups. The organic chain may include side chains, such as alkyl, alkenyl, or aryl side chains. The organic chain may include one or more functional groups such as ether groups, carboxy groups, hydroxy groups, halide groups, etc. The organic chain may include one or more heteroatoms along the chain, for instance the chain may include one or more polyethylene glycol units. The organic chain may be a polyethylene glycol chain and/or one or more cyclic moieties, such as a heterocycle or a cycloalkane.

The organic chain may have a length of 1 to 40 atoms. The organic chain may have a length of 1 to 15 atoms. The organic chain may have a length of at least 3 atoms or at least 5 atoms.

In the example preparation above, the RIF block is 3-formylrifamycin SV, also known as rifaldehyde. In other examples, the RIF block may be any rifamycin or rifamycin derivative that is suitable for reacting with (i.e., coupling to) the first functional group of the linker precursor. The RIF block could for instance be based on the examples shown in Table 1 or modified versions of the examples shown in Table 1.

In some examples, the linker precursor may be modified prior to reaction with the RIF block. The linker precursor may for example be modified with 1-Boc-4-piperidone via reductive amination. Such modified linker precursors may be used to form example compounds 2A to 2D of Table 2. An example modification of a linker precursor with 1-Boc-4-piperidone is shown in Scheme 2 below.

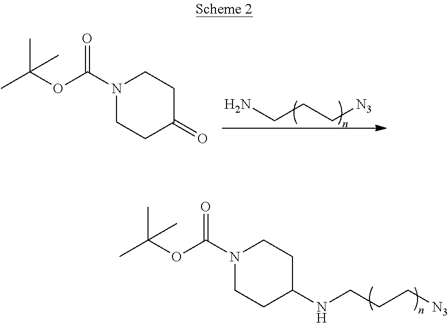

Example intermediates are shown in Table 4 below.

TABLE 4

| ID | Structure |
|---|---|
| 4A | 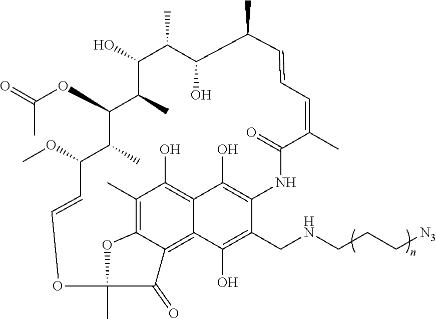 |
| | wherein n is from 0 to 10 |
| 4B | 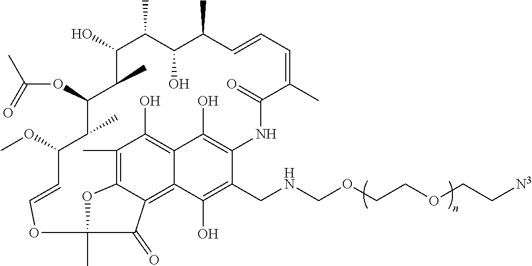 |
| | wherein n is from 1 to 10 |
| 4C | 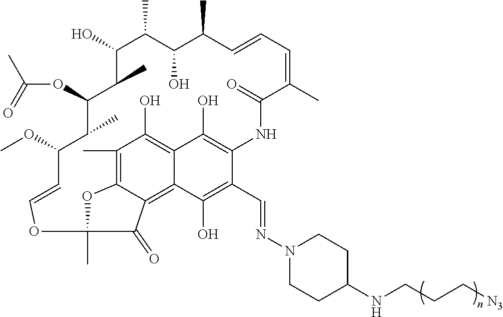 |
| | wherein n is from 1 to 10 |
| 4D | 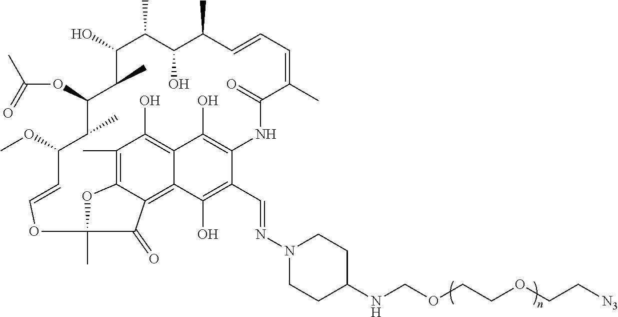 |
| | wherein n is from 1 to 10 |

### Preparation of the NUC Block

In some examples, the nucleobase analogue or nucleoside analogue of the NUC block are modified to facilitate the reaction with the linker precursor. An example modification of a nucleobase analogue of a NUC block is shown in Scheme 3 below. Such a modification could be used in the synthesis of examples 2A, 2B, 2E and 2F of Table 2.

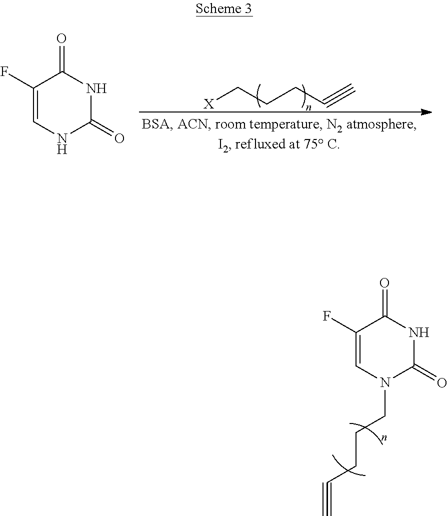

In the example preparation of Scheme 3, a nucleobase analogue 5-Fluorouracil (1 equivalent) was added to a round bottom flask of 10 mL acetonitrile. Next, bis(trimethylsilyl) acetamide (BSA) (2.4 equivalents) was added to the suspension and stirred at room temperature under N₂ atmosphere until the solution was clear. An alkyne with an electrophilic centre (10 equivalents) and a catalytic amount of iodine were added to the clear solution that was heated at reflux (75° C.) until the starting materials were determined to be consumed by thin layer chromatography. The solution was cooled to room temperature and concentrated under reduced pressure, distilled water was added to the residue and then extracted with ethyl acetate. The organic layer was collected, dried with magnesium sulphate, filtered and evaporated to dryness.

In the example preparation above, the nucleobase analogue is modified by an alkyne with an electrophilic centre. In other examples, the nucleobase analogue could be modified with any reagent with a first functional group that is suitable for reacting with (i.e., coupling to) the nucleobase analogue and with a second functional group that is suitable for reacting with (i.e., coupling to) the second functional group of the linker precursor.

In some examples, the alkyne with an electrophilic centre is a haloalkyne (such as 5-chloro-1-pentyne or 4-bromo-1-butyne) or an alkynoic acid, such as pent-4-ynoic acid.

The alkyne could be of different lengths. For instance, the value n in the alkyne of Scheme 1 may be between 0 and 5.

An example modification of a nucleoside analogue of an NUC block is shown in Scheme 4 below. In the example of Scheme 4, the nucleoside analogue is emtricitabine and the nucleoside analogue is modified by reaction with pent-4-ynoic acid. Such a modification could be used in the synthesis of examples 2C, 2D, 2G and 2H of Table 2.

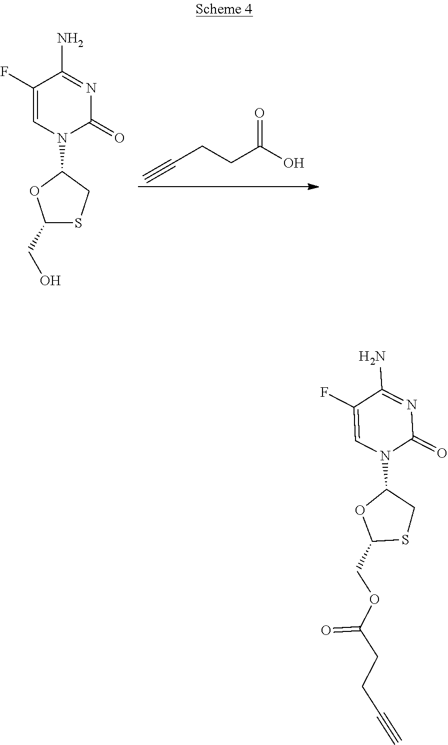

In the example preparation above, the nucleoside analogue is modified by pent-4-ynoic acid. In other examples, the nucleoside analogue could be modified with any reagent with a first functional group that is suitable for reacting with (i.e., coupling to) the nucleoside analogue and with a second functional group that is suitable for reacting with (i.e., coupling to) the second functional group of the linker precursor.

Reaction of the Intermediate with the NUC Block

The NUC block can be reacted with the intermediate to form the compound. An example reaction between an intermediate and a NUC block is shown below in Scheme 5. In this example the intermediate is the product of Scheme 1 and the NUC block is the product of Scheme 3.

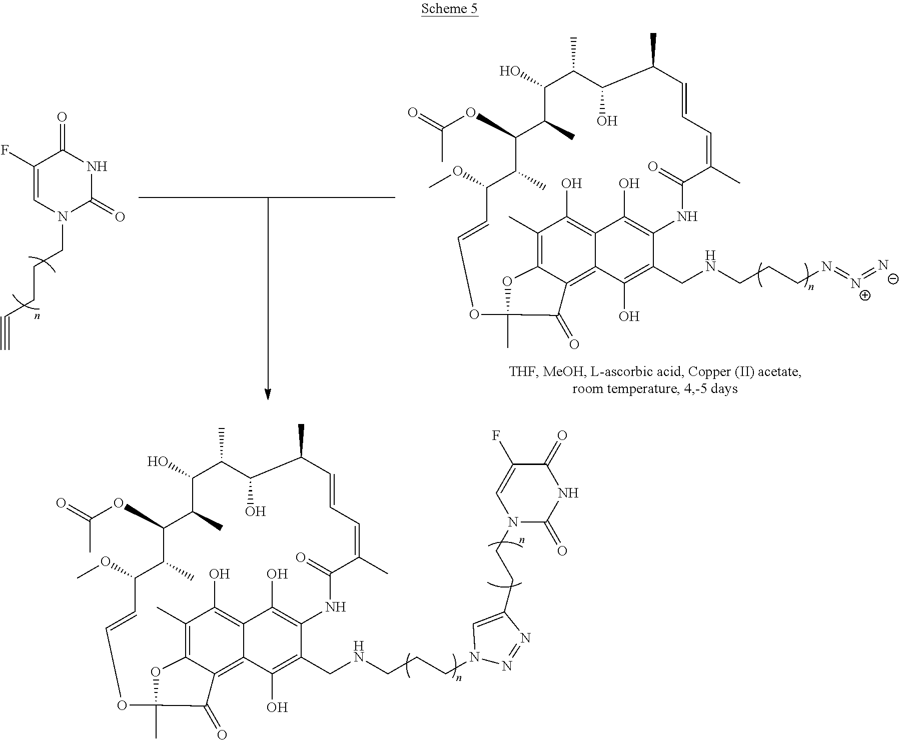

The intermediate (5 equivalents) was added to a round bottom flask of tetrahydrofuran (THF) and methanol (MeOH). To this solution, the NUC block (5 equivalents) was added as well as L-ascorbic acid (3.8 equivalents) and the catalyst copper (II) acetate (1 equivalent). The reaction was stirred at room temperature until thin layer chromatography confirmed the starting materials had been consumed. The solution was evaporated to dryness and then extracted using ethyl acetate. The organic layers were combined and washed with brine, dried with magnesium sulphate, and filtered before being concentrated under reduced pressure.

### Preparation of Compound 3A

Compound 3A of Table 3 was prepared according to the following procedure.

Step 1—3-azido-1-propanamine (1 equivalent) was added to a round bottom flask containing 10 mL of dichloromethane in which 3-formyl rifamycin SV (1 equivalent) was previously dissolved. The solution was then treated with sodium triacetoxyborohydride (1.4 equivalents), and stirred at room temperature under an N₂ atmosphere, until the consumption of the starting materials was observed by thin layer chromatography (4.5 hours). At which point the reaction was neutralised through the addition of 10 mL 10% sodium bicarbonate. Ethyl acetate was then used to extract the product, and the organic layers were collected and washed with brine. Magnesium sulphate was used to dry the extracts, which was then filtered and concentrated under reduced pressure to provide an intermediate. 1H-NMR (600 MHz, CHLOROFORM-D) δ 10.66 (s, 1H), 9.08 (s, 1H), 6.56 (s, 2H), 6.37 (d, J=16.2 Hz, 2H), 6.21 (s, 1H), 6.00 (s, 1H), 5.31-5.40 (2H), 4.98 (d, J=15.6 Hz, 1H), 3.75 (d, J=8.9 Hz, 1H), 3.72 (t, J=6.0 Hz, 1H), 3.60 (s, 1H), 3.59 (s, 1H), 3.54 (s, 1H), 3.37 (s, 2H), 3.06 (s, 2H), 2.30 (dd, J=13.4, 7.6 Hz, 4H), 2.09 (s, 2H), 2.06 (s, 3H), 2.04 (d, J=3.8 Hz, 3H), 1.80 (d, J=6.0 Hz, 4H), 1.68 (s, 1H), 1.60 (s, 1H), 1.49 (s, 2H), 1.02 (d, J=7.0 Hz, 3H), 0.88 (d, J=7.0 Hz, 4H), 0.61 (d, J=6.9 Hz, 3H). 13C-NMR (151 MHz, CHLOROFORM-D) δ 195.8, 194.0, 177.6, 172.1, 166.0, 160.0, 154.2, 142.0, 135.4, 128.8, 118.0, 112.6, 110.1, 108.5, 89.4, 84.8, 74.2, 57.1, 49.0, 47.1, 42.5, 38.9, 37.3, 33.1, 29.5, 21.9, 20.9, 17.2, 11.0, 8.6, 8.3, 7.4.

Step 2—5-Fluorouracil (1 equivalent) was added to a round bottom flask of 10 mL acetonitrile. Next, bis(trimethylsilyl) acetamide (2.4 equivalents) was added to the suspension and stirred at room temperature under N₂ atmosphere until the solution was clear. 4-Bromo-1-butyne (10 equivalents) and a catalytic amount of iodine were added to the clear solution and were heated at reflux (75° C.) until the starting materials were determined to be consumed by thin layer chromatography (22 hours). The solution was cooled to room temperature and concentrated under reduced pressure, distilled water was added to the residue and then extracted with ethyl acetate, the organic layer was collected, dried with magnesium sulphate, filtered and evaporated to dryness to provide a NUC block. 1H-NMR (600 MHz, CHLOROFORM-D) δ 7.07 (s, 1H), 3.51 (t, J=7.2 Hz, 2H), 3.07 (t, J=7.1 Hz, 2H). LC-MS: m/z (ES+) 183.2 [M+H]+.

Step 3—The intermediate produced in step 1 (5 equivalents) was added to a round bottom flask of tetrahydrofuran (THF) and methanol (MeOH). To this solution, the NUC block produced in step 2 (5 equivalents) was added as well as L-ascorbic acid (3.8 equivalents) and the catalyst copper (II) acetate (1 equivalent). The reaction was stirred at room temperature until thin layer chromatography confirmed the starting materials had been consumed (4 days). The solution was evaporated to dryness and then extracted using ethyl acetate. The organic layers were combined and washed with brine, dried with magnesium sulphate, and filtered before being concentrated under reduced pressure to provide the compound 3A. 1H-NMR (600 MHz, CHLOROFORM-D) δ 11.93 (s, 1H), 10.75-10.71 (m, 1H), 7.47 (d, J=13.1 Hz, 1H), 6.62 (d, J=32.9 Hz, 1H), 6.12 (d, J=12.4 Hz, 1H), 5.74 (dd, J=16.1, 6.8 Hz, 1H), 5.27-5.15 (m, 4H), 3.82 (q, J=3.7 Hz, 1H), 3.59 (d, J=9.3 Hz, 1H), 3.50-3.39 (m, 7H), 3.14 (s, 3H), 2.98 (s, 3H), 2.61 (d, J=2.6 Hz, 1H), 2.43 (q, J=6.9 Hz, 1H), 2.27 (s, 3H), 2.07 (s, 3H), 1.90-1.86 (m, 2H), 1.73 (s, 3H), 1.18 (s, 3H), 1.06 (d, J=6.9 Hz, 3H), 0.91 (d, J=6.9 Hz, 3H), 0.73 (d, J=6.7 Hz, 3H), 0.60 (d, J=6.9 Hz, 3H). 13C-NMR (151 MHz, CHLOROFORM-D) δ 195.6, 193.5, 177.4, 172.6, 166.4, 160.8, 154.5, 141.4, 136.7, 122.6, 117.5, 113.6, 111.3, 107.5, 89.1, 84.9, 84.1, 74.7, 73.1, 56.5, 56.2, 48.0, 47.1, 40.7, 39.0, 37.7, 29.7, 23.4, 21.5, 20.9, 18.8, 14.1, 10.7, 8.6, 7.6. HR-MS: found 991.4335 calculated for C₄₉H₆₂FN₇O₁₄ 991.4338.

### Preparation of Compound 3B

Compound 3B of Table 3 was prepared according to the following procedure.

Step 1—11-Azido-3,6,9-trioxaundecan-1-amine (1 equivalent) was added to a round bottom flask containing 10 mL of dichloromethane in which 3-formyl rifamycin SV (1 equivalent) was previously dissolved. The solution was then treated with sodium triacetoxyborohydride (1.4 equivalents), and stirred at room temperature under an N₂ atmosphere, until the consumption of the starting materials was observed by thin layer chromatography (5 hours). At which point the reaction was neutralised through the addition of 10 mL 10% sodium bicarbonate. Ethyl acetate was then used to extract the product, and the organic layers were collected and washed with brine. Magnesium sulphate was used to dry the extracts, which was then filtered and concentrated under reduced pressure to provide an intermediate. 1H-NMR (600 MHz, CHLOROFORM-D) δ 10.68 (d, J=3.9 Hz, OH), 6.34 (d, J=11.1 Hz, 1H), 6.16 (dd, J=12.5, 0.9 Hz, 1H), 5.17-5.11 (m, 1H), 4.97 (dd, J=30.9, 10.6 Hz, 1H), 3.76 (d, J=9.0 Hz, 1H), 3.67-3.61 (m, 12H), 3.50-3.48 (m, 1H), 3.36 (t, J=5.0 Hz, 2H), 3.09-3.01 (m, 5H), 2.41-2.35 (m, 1H), 2.26-2.17 (m, 3H), 2.10 (s, 2H), 2.08-2.00 (m, 4H), 1.81 (d, J=10.3 Hz, 3H), 1.75-1.69 (m, 1H), 1.61-1.55 (m, 1H), 1.03 (t, J=6.6 Hz, 3H), 0.84 (d, J=7.2 Hz, 3H), 0.61 (d, J=6.7 Hz, 3H).

Step 2—5-Fluorouracil (1 equivalent) was added to a round bottom flask of 10 mL acetonitrile. Next, bis(trimethylsilyl) acetamide (2.4 equivalents) was added to the suspension and stirred at room temperature under N₂ atmosphere until the solution was clear. 4-Bromo-1-butyne (10 equivalents) and a catalytic amount of iodine were added to the clear solution and were heated at reflux (75° C.) until the starting materials were determined to be consumed by thin layer chromatography (22 hours). The solution was cooled to room temperature and concentrated under reduced pressure, distilled water was added to the residue and then extracted with ethyl acetate, the organic layer was collected, dried with magnesium sulphate, filtered and evaporated to dryness to provide a NUC block. 1H-NMR (600 MHz, CHLOROFORM-D) δ 7.07 (s, 1H), 3.51 (t, J=7.2 Hz, 2H), 3.07 (t, J=7.1 Hz, 2H). LC-MS: m/z (ES+) 183.2 [M+H]+.

Step 3—The intermediate produced in step 1 (5 equivalents) was added to a round bottom flask of tetrahydrofuran (THF) and methanol (MeOH). To this solution, the NUC block produced in step 2 (5 equivalents) was added as well as L-ascorbic acid (3.8 equivalents) and the catalyst copper (II) acetate (1 equivalent). The reaction was stirred at room temperature until thin layer chromatography confirmed the starting materials had been consumed (5 days). The solution was evaporated to dryness and then extracted using ethyl acetate. The organic layers were combined and washed with brine, dried with magnesium sulphate, and filtered before being concentrated under reduced pressure to provide the compound 3B. 1H-NMR (600 MHz, CHLOROFORM-D) δ 12.01 (s, 1H), 10.84-10.80 (m, 1H), 7.59 (d, J=13.1 Hz, 1H), 7.06 (d, J=22.7 Hz, 1H), 6.12 (d, J=12.4 Hz, 1H), 5.73 (dd, J=16.0, 6.7 Hz, 1H), 5.27-5.16 (m, 4H), 3.83-3.81 (m, 1H), 3.59 (d, J=216.0 Hz, 20H), 3.16 (s, 3H), 3.04 (d, J=10.3 Hz, 2H), 2.99 (s, 3H), 2.44 (q, J=6.8 Hz, 1H), 2.35 (q, J=7.4 Hz, 1H), 2.27 (s, 3H), 2.02 (d, J=68.7 Hz, 6H), 1.73 (s, 3H), 1.17 (s, 3H), 1.06 (d, J=6.9 Hz, 3H), 0.92 (d, J=6.9 Hz, 3H), 0.87 (q, J=7.1 Hz, 4H), 0.72 (d, J=6.7 Hz, 3H), 0.60 (d, J=7.2 Hz, 3H). HR-MS: found 1095.4809 calculated for C₅₃H₇₀FN₇O₁₇ 1095.4812.

### Preparation of Compound 3C

Compound 3C of Table 3 was prepared according to the following procedure.

Step 1—11-Azido-3,6,9-trioxaundecan-1-amine (1 equivalent) was added to a round bottom flask containing 10 mL of dichloromethane in which 3-formyl rifamycin SV (1 equivalent) was previously dissolved. The solution was then treated with sodium triacetoxyborohydride (1.4 equivalents), and stirred at room temperature under an N₂ atmosphere, until the consumption of the starting materials was observed by thin layer chromatography (5 hours). At which point the reaction was neutralised through the addition of 10 mL 10% sodium bicarbonate. Ethyl acetate was then used to extract the product, and the organic layers were collected and washed with brine. Magnesium sulphate was used to dry the extracts, which was then filtered and concentrated under reduced pressure to provide an intermediate. 1H-NMR (600 MHz, CHLOROFORM-D) δ 10.68 (d, J=3.9 Hz, OH), 6.34 (d, J=11.1 Hz, 1H), 6.16 (dd, J=12.5, 0.9 Hz, 1H), 5.17-5.11 (m, 1H), 4.97 (dd, J=30.9, 10.6 Hz, 1H), 3.76 (d, J=9.0 Hz, 1H), 3.67-3.61 (m, 12H), 3.50-3.48 (m, 1H), 3.36 (t, J=5.0 Hz, 2H), 3.09-3.01 (m, 5H), 2.41-2.35 (m, 1H), 2.26-2.17 (m, 3H), 2.10 (s, 2H), 2.08-2.00 (m, 4H), 1.81 (d, J=10.3 Hz, 3H), 1.75-1.69 (m, 1H), 1.61-1.55 (m, 1H), 1.03 (t, J=6.6 Hz, 3H), 0.84 (d, J=7.2 Hz, 3H), 0.61 (d, J=6.7 Hz, 3H).

Step 2—5-Fluorouracil (1 equivalent) was added to a round bottom flask of 10 mL acetonitrile. Next, bis(trimethylsilyl) acetamide (2.4 equivalents) was added to the suspension and stirred at room temperature under N₂ atmosphere until the solution was clear. 5-Chloro-1-pentyne (10 equivalents) and a catalytic amount of iodine were added to the clear solution and were heated at reflux (75° C.) until the starting materials were determined to be consumed by thin layer chromatography (24 hours). The solution was cooled to room temperature and concentrated under reduced pressure, distilled water was added to the residue and then extracted with ethyl acetate, the organic layer was collected, dried with magnesium sulphate, filtered and evaporated to dryness to provide a NUC block. 1H-NMR (600 MHz, CHLOROFORM-D) δ 6.91 (s, 1H), 3.57 (td, J=6.5, 1.0 Hz, 2H), 2.71-2.68 (m, 2H), 2.06-2.01 (m, 2H). LC-MS: m/z (ES+) 197.2 [M+H]+.

Step 3—The intermediate produced in step 1 (5 equivalents) was added to a round bottom flask of tetrahydrofuran (THF) and methanol (MeOH). To this solution, the NUC block produced in step 2 (5 equivalents) was added as well as L-ascorbic acid (3.8 equivalents) and the catalyst copper (II) acetate (1 equivalent). The reaction was stirred at room temperature until thin layer chromatography confirmed the starting materials had been consumed (5 days). The solution was evaporated to dryness and then extracted using ethyl acetate. The organic layers were combined and washed with brine, dried with magnesium sulphate, and filtered before being concentrated under reduced pressure to provide the compound 3C. 1H-NMR (600 MHz, CHLOROFORM-D) δ 12.01 (s, 1H), 10.84-10.80 (m, 1H), 7.60 (d, J=13.1 Hz, 1H), 7.13-7.08 (m, 1H), 6.12 (d, J=12.4 Hz, 1H), 5.73 (dd, J=16.1, 6.8 Hz, 1H), 5.27-5.16 (m, 3H), 3.82 (q, J=3.6 Hz, 1H), 3.59 (d, J=214.4 Hz, 21H), 3.16 (s, 3H), 3.04 (d, J=10.6 Hz, 1H), 2.98 (s, 3H), 2.44 (q, J=6.8 Hz, 1H), 2.27 (s, 3H), 1.99-2.11 (8H), 1.73 (s, 3H), 1.17 (s, 3H), 1.06 (d, J=6.9 Hz, 3H), 0.92 (d, J=6.9 Hz, 3H), 0.72 (d, J=6.7 Hz, 3H), 0.60 (d, J=7.2 Hz, 3H). 13C-NMR (151 MHz, CHLOROFORM-D) δ 195.8, 193.5, 180.2, 176.9, 173.4, 172.6, 166.4, 156.1, 141.4, 141.0, 137.1, 122.9, 117.4, 113.3, 111.4, 110.0, 107.5, 101.9, 89.4, 84.8, 84.0, 78.1, 77.7, 74.7, 73.2, 70.8, 70.7, 70.7, 70.5, 69.9, 56.5, 56.2, 50.6, 48.9, 40.7, 39.0, 37.7, 34.9, 31.9, 29.7, 23.4, 21.5, 20.9, 18.8, 10.7, 8.6, 7.6. HR-MS: found 1109.4959 calculated for C₅₄H₇₂FN₇O₁₇ 1109.4968.

### Experimental Data

The in vitro antitubercular activities of a selection of compounds of the disclosure against M. tuberculosis H37Rv (ATCC 25618 Wild-type), HN-878 (wild-type), rifampicin resistant strain RpoB_{S522L} (H37Rv), and rifampicin-resistant strain RpoB_{S531L} are shown in Table 5 below. The compounds shown in Table 5 include compounds 3A, 3B and 3C of Table 3, along with rifampicin and isoniazid.

TABLE 5

| | Mycobacterium tuberculosis strain MIC₉₀ (μM) | | | |
|---|---|---|---|---|
| | | | RpoB_{S522L} | |
| Compound | H37Rv | HN-878 | H37Rv | RpoB_{S531L} |
| 3A | 0.0039 | 0.059 ± 0.010 | 0.31 | 0.22 |
| 3B | 0.0039 | 0.096 ± 0.064 | 0.32 | 0.23 |
| 3C | 0.0034 | 0.070 ± 0.042 | 0.47 | 0.21 |
| Rifampicin | 0.0045 | 0.02 | >8.0 | >8.0 |
| Isoniazid | 0.28 | 0.29 | 0.29 | 0.55 |

MIC₉₀ is defined as the concentration required to inhibit growth of M. tuberculosis in liquid medium by 90% after 5 days. The screening was conducted in triplicate. The MIC of a compound was determined by measuring bacterial growth after 5 days in the presence of test compounds. Compounds were prepared as 10-point two-fold serial dilutions in DMSO and diluted into 7H9-Tw-OADC medium (recipe: 4.7 g/L 7H9 base broth, 0.05% w/v Tween 80, 10% v/v OADC supplement) in 96-well plates with a final DMSO concentration of 2%. Each plate included assay controls for background (medium/DMSO only, no bacterial cells), zero growth (100 μM rifampicin) and maximum growth (DMSO only). Plates were inoculated with M. tuberculosis and incubated for 5 days: growth was measured by OD590 and fluorescence (Ex 560/Em 590) using a BioTek™ Synergy H4 plate reader. A dose response curve was plotted as % growth and fitted using the Levenberg-Marquardt algorithm. Concentrations that resulted in 50% and 90% inhibition of growth were determined.

As shown clearly in Table 5, compounds 3A, 3B and 3C exhibit significantly improved activity against rifampicin resistant strains, when compared to rifampicin. The compounds according to the disclosure can therefore be used as to treat infection or prevent infection where antibiotic resistance may be present.

The in vitro antistaphylococcal activities of a selection of compounds of the disclosure against S. aureus NCTC 12493 are shown in Table 6 below. The compounds shown in Table 6 include compounds 3A and 3B of Table 3, along with rifampicin and fluorouracil (5-FU).

TABLE 6

S. aureus NCTC 12493

| Compound | MIC (μg/mL) | MBC (μg/mL) |
|---|---|---|
| 3A | 1 | 1 |
| 3B | 2 | 2 |
| Rifampicin | 0.25 | 4 |
| 5-FU | 2 | 16 |

The MIC value of Table 6 is the minimum inhibitory concentration, which is the lowest concentration of an antibacterial agent that inhibits the visible in vitro growth of a microorganism. The MBC value of Table 6 is the minimum bactericidal concentration, which is identified by determining the lowest concentration of antibacterial agent that reduces the viability of the initial bacterial inoculum by ≥99.9%. The screening of Table 6 was carried out using the broth microdilution method (CLSI2009. Methods fordilution antimicrobial susceptibility tests for bacteria that grow aerobically. Approved standard, 8th ed M7-A8. CLSI, Wayne, PA). MBC values were determined by subculturing 10 μL from each well without visible bacterial growth on Mueller-Hinton agar plates. After 24 h of incubation at 37° C., the dilution yielding three colonies or fewer was scored as the MBC, as described by the Clinical and Laboratory Standards Institute (CLSI) for starting inocula of 1×105 CFU/mL (NCCLS 1999. Methods for determining bactericidal activity of antimicrobial agents. Approved guideline. M26-A. CLSI, Wayne, PA).

If the MIC of a compound is closer to the MBC, the compound is considered more bactericidal. As shown in Table 6, the MIC and MBC values for compounds 3A and 3B of Table 6 are the same, thus denoting high bactericidal activity of these bifunctional compounds relative to rifampicin and fluorouracil (5-FU). The MBC values of rifampicin and fluorouracil (5-FU) are 16 and 8 times higher than their MIC values, respectively. Furthermore, compound 3A exhibited the lowest MBC value among the compounds tested.

There is thus described compounds according to examples of the disclosure with a number of advantages as detailed above.

Although embodiments of the present invention have been described in the preceding paragraphs with reference to various examples, it should be appreciated that modifications to the examples given can be made without departing from the scope of the invention as claimed. For instance, a different rifamycin or rifamycin derivative could be used, a different nucleobase analogue or nucleoside analogue could be used, and/or a different linker could be used. The compounds could be produced using a different method, for instance using different click chemistry reactions to combine the RIF unit, the linker precursor and the NUC unit.

The term ‘comprise’ is used in this document with an inclusive not an exclusive meaning. That is any reference to X comprising Y indicates that X may comprise only one Y or may comprise more than one Y. If it is intended to use ‘comprise’ with an exclusive meaning then it will be made clear in the context by referring to “comprising only one” or by using “consisting”.

In this description, reference has been made to various examples. The description of features or functions in relation to an example indicates that those features or functions are present in that example. The use of the term ‘example’ or ‘for example’ or ‘can’ or ‘may’ in the text denotes, whether explicitly stated or not, that such features or functions are present in at least the described example, whether described as an example or not, and that they can be, but are not necessarily, present in some of or all other examples. Thus ‘example’, ‘for example’, ‘can’ or ‘may’ refers to a particular instance in a class of examples. A property of the instance can be a property of only that instance or a property of the class or a property of a sub-class of the class that includes some but not all of the instances in the class. It is therefore implicitly disclosed that a feature described with reference to one example but not with reference to another example, can where possible be used in that other example as part of a working combination but does not necessarily have to be used in that other example.

Features described in the preceding description may be used in combinations other than the combinations explicitly described above.

Although functions have been described with reference to certain features, those functions may be performable by other features whether described or not.

Although features have been described with reference to certain examples, those features may also be present in other examples whether described or not.

The term ‘a’ or ‘the’ is used in this document with an inclusive not an exclusive meaning. That is any reference to X comprising a/the Y indicates that X may comprise only one Y or may comprise more than one Y unless the context clearly indicates the contrary. If it is intended to use ‘a’ or ‘the’ with an exclusive meaning then it will be made clear in the context. In some circumstances the use of ‘at least one’ or ‘one or more’ may be used to emphasis an inclusive meaning but the absence of these terms should not be taken to infer any exclusive meaning.

The presence of a feature (or combination of features) in a claim is a reference to that feature or (combination of features) itself and also to features that achieve substantially the same technical effect (equivalent features). The equivalent features include, for example, features that are variants and achieve substantially the same result in substantially the same way. The equivalent features include, for example, features that perform substantially the same function, in substantially the same way to achieve substantially the same result.

In this description, reference has been made to various examples using adjectives or adjectival phrases to describe characteristics of the examples. Such a description of a characteristic in relation to an example indicates that the characteristic is present in some examples exactly as described and is present in other examples substantially as described.

Whilst endeavoring in the foregoing specification to draw attention to those features believed to be of importance it should be understood that the applicant may seek protection via the claims in respect of any patentable feature or combination of features hereinbefore referred to whether or not emphasis has been placed thereon.

1. A compound with the general structure of formula I
R¹-L-R² (Formula I)
wherein
R¹ is a rifamycin or a rifamycin derivative;
L is a linker;
R² is a nucleobase analogue or a nucleoside analogue; and
the compound is for use as a medicament.

2. A compound according to claim 1, wherein the nucleobase analogue or nucleoside analogue comprises a purine analogue moiety or a pyrimidine analogue moiety.

3. A compound according to claim 1, wherein the nucleobase analogue or nucleoside analogue is thioguanine, 6-mercaptopurine, 6-thioguanosine, 5-fluorouracil, 5-fluorocytosine, emtricitabine, 2′-deoxycytidine, floxuridine, 5-fluorouridine, trifluridine, 2′, 3′-dideoxyinosine, gemcitabine, zidovudine or idoxuridine.

4. A compound according to claim 1, wherein the nucleobase analogue or nucleoside analogue is 5-fluorouracil or emtricitabine.

5. A compound according to claim 1, wherein the rifamycin or rifamycin derivative has the general formula II or the general formula III. 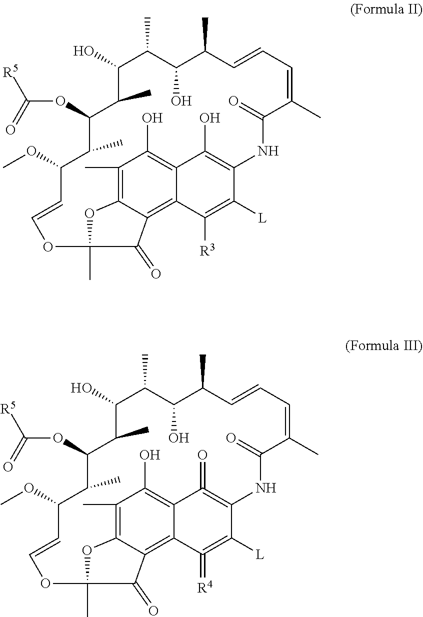
wherein
R³ is selected from an OH group, an NH₂ group, a carboxymethoxy group, or a heterocycle formed with the linker;
R⁴ is selected from an O, an NH group, or a heterocycle formed with the linker;
R⁵ is selected from a CH₃ group, a secondary amine or a tertiary amine; and
L indicates the attachment to the linker.

6. A compound according to claim 1, wherein the rifamycin or rifamycin derivative has the general formula IV 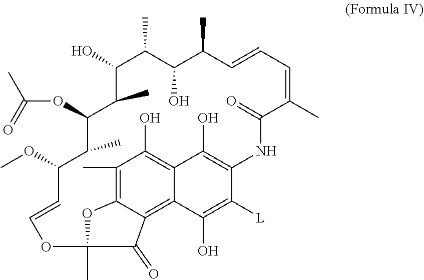
wherein:
L indicates the attachment to the linker.

7. A compound according to claim 1, wherein the linker is configured to substantially prevent steric hindrance between R¹ and R².

8. A compound according to claim 1, wherein the linker is an organic chain with a length of 3 to 50 atoms.

9. A compound according to claim 1, wherein the linker is an organic chain with a length of 5 to 30 atoms.

10. A compound according to claim 1, wherein the linker is an organic chain with a length of at least 12 atoms.

11. A compound according to claim 1, wherein the linker comprises:
a linear or branched alkyl chain; or
an ethylene glycol chain.

12. A compound according to claim 1, wherein the linker has the general formula V
R¹-L¹-L²-L³-R² (Formula V)
wherein
R¹ indicates attachment to R¹;
R² indicates attachment to R²;
L¹ is selected from: 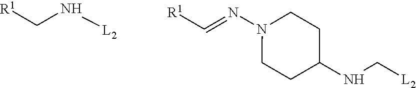
wherein R¹ indicates attachment to R¹ and L² indicates attachment to L²
L² is an organic chain with a length of 1 to 50 atoms; and
L³ is selected from: 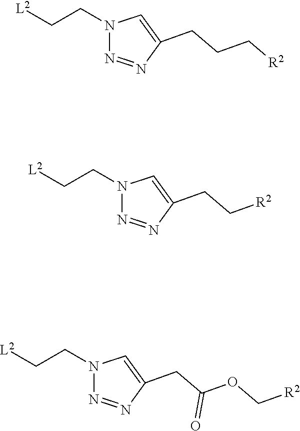
wherein L² indicates attachment to L² and R² indicates attachment to R².

13. A compound according to claim 11, wherein L² is
a linear or branched alkyl chain; or
an ethylene glycol chain.

14. A compound according to claim 1, wherein the compound is selected from: 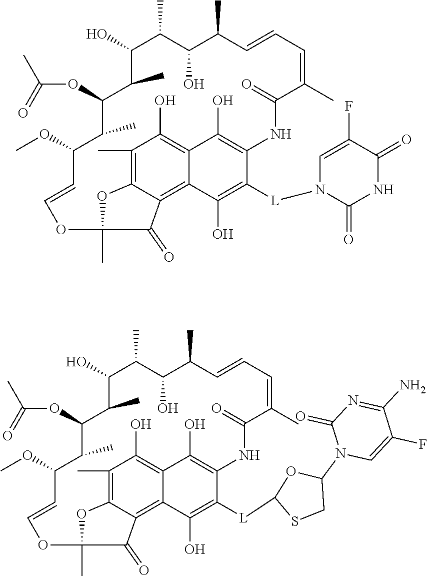
wherein L is the linker.

15. A compound according to claim 1, wherein the compound is selected from: 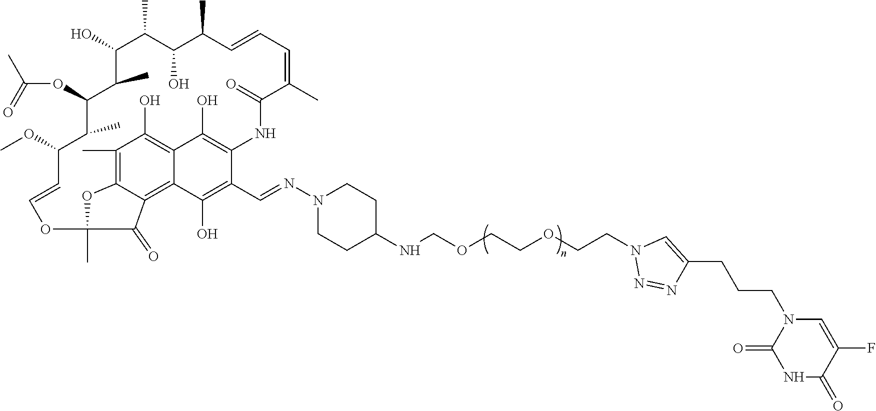
wherein n is from 1 to 10 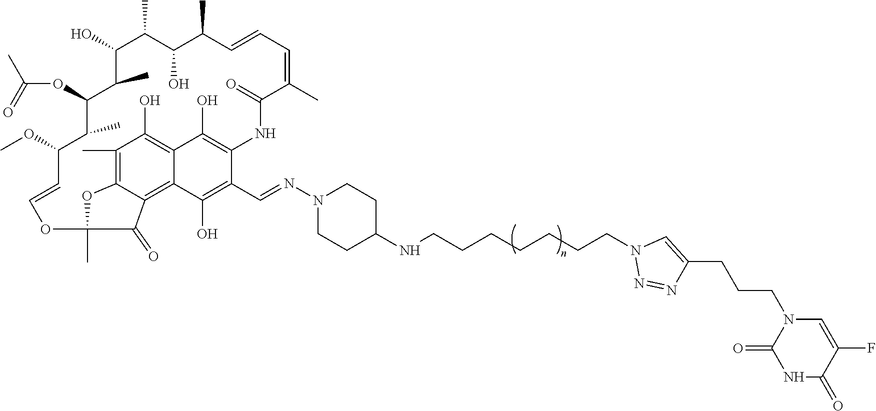
wherein n is from 0 to 10 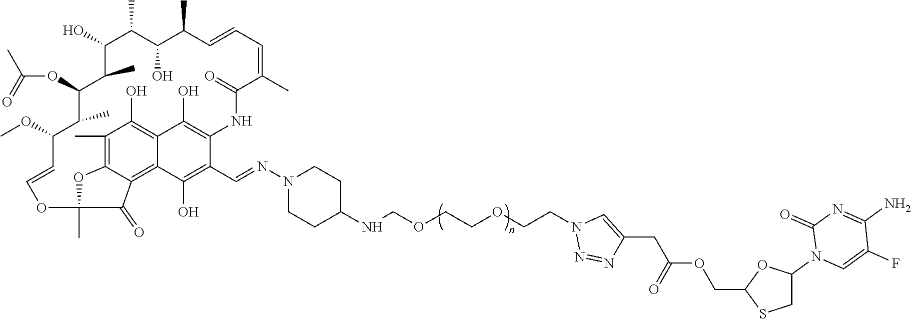
wherein n is from 1 to 10 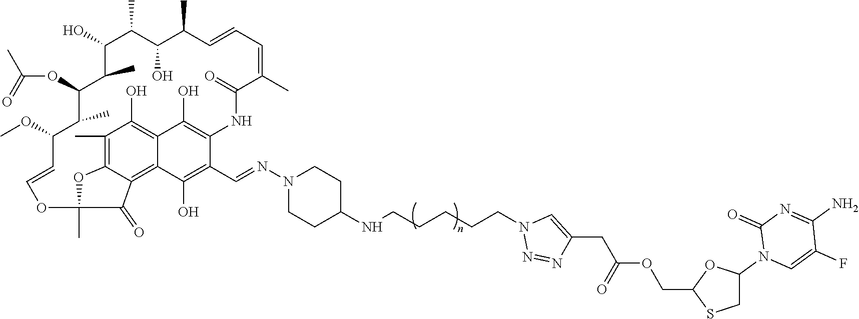
wherein n is from 0 to 10 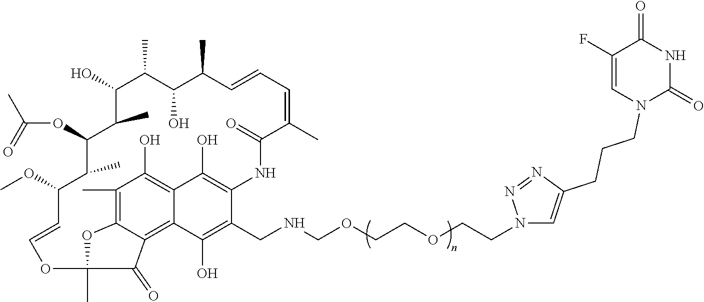
wherein n is from 1 to 10 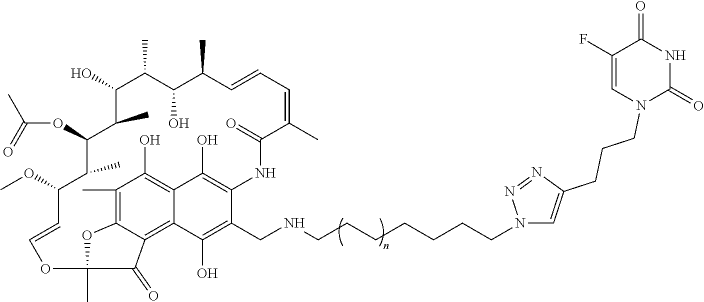
wherein n is from 0 to 10 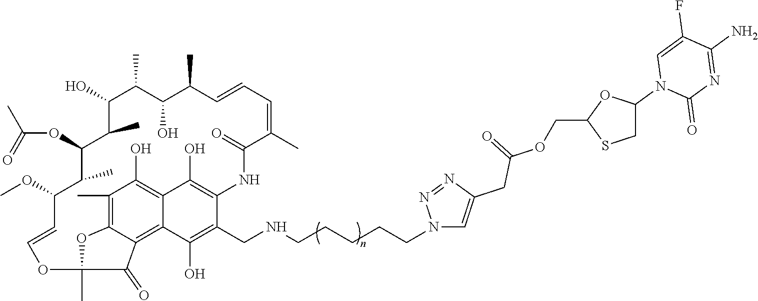
wherein n is from 0 to 10 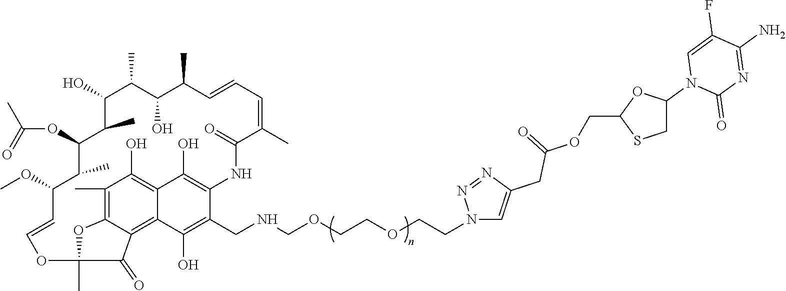
wherein n is from 1 to 10.

16. A compound according to claim 1, wherein the compound is selected from:

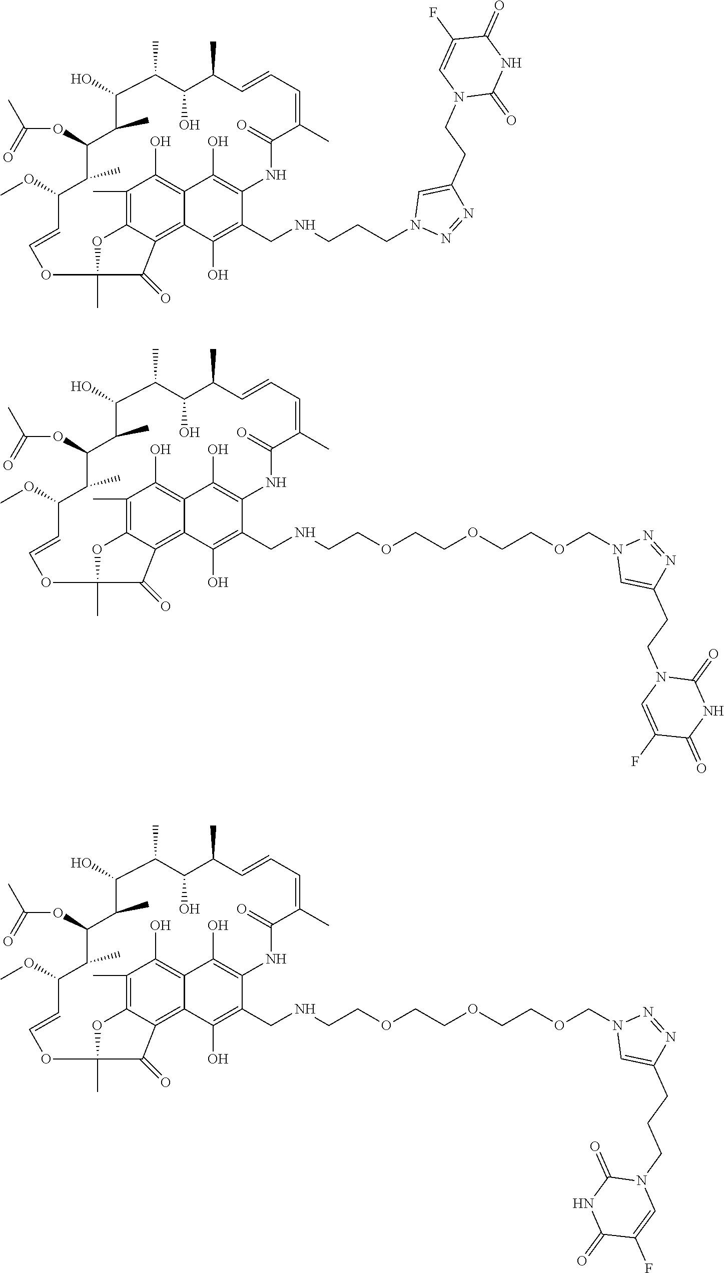

17. A compound according to claim 1 for use in the treatment of an infection or prevention of an infection.

18. A pharmaceutical composition which comprises a compound according to any of the preceding claims, or a pharmaceutically acceptable salt, solvate or prodrug thereof, in association with a pharmaceutically acceptable diluent or carrier.

19. A method of treating or preventing an infection in an individual, the method comprising the step of administering to the individual a therapeutically effective amount of the compound of claim 1.
